(19) United States
(12) Patent Application Publication
(10) Pub. No.: US 2019/0282213 A1
(43) Pub. Date: Sep. 19, 2019

(54) SYSTEMS AND METHODS FOR MOTION-BASED CONTROL OF ULTRASOUND IMAGES

(71) Applicant: EchoNous, Inc., Redmond, WA (US)

(72) Inventors: Brandon S. Cheung, San Francisco, CA (US); Ramachandra Pailoor, Woodinville, WA (US); Eung-Hun Kim, Bothell, WA (US); Bradley Scott Melmon, Seattle, WA (US); Greg Nieminen, Bothell, WA (US); Shelby Brunke, Sammamish, WA (US); Nidhi Jaiswal, Redmond, WA (US); Qianying Miao, Redmond, WA (US)

(21) Appl. No.: 16/355,257

(22) Filed: Mar. 15, 2019

Related U.S. Application Data
(60) Provisional application No. 62/644,193, filed on Mar. 16, 2018.

(51) Int. Cl. A61B 8/08; A61B 8/00

(52) U.S. Cl. CPC A61B 8/5276; A61B 8/4245; A61B 8/467; A61B 8/463; A61B 8/4444

(57) ABSTRACT

Systems and methods for controlling parameters associated with ultrasound images displayed on a computing device, based on sensed motion of the computing device, are provided herein. One such system includes an ultrasound probe and a computing device coupled to the ultrasound probe and operable to receive ultrasound signals from the ultrasound probe. The computing device includes a motion sensor that senses motion of the computing device, a display that displays ultrasound images associated with the ultrasound signals received from the ultrasound probe, and an image display controller that controls at least one parameter associated with the displayed ultrasound images based on the sensed motion.

### BACKGROUND

### Technical Field

The present disclosure pertains to ultrasound systems, and more particularly to ultrasound systems and methods for controlling a parameter of a displayed ultrasound image based on a sensed motion of a handheld computing device.

### Description of the Related Art

In conventional ultrasound imaging systems, a healthcare professional holds an ultrasound probe in a desired position, e.g., on a patient's body, and may view acquired ultrasound images on a computer screen that is typically located in a fixed position, such as on an ultrasound cart or other such equipment. Input devices, such as a keyboard, mouse, buttons, track-pad, track-ball or the like may be provided on the cart and allow the user to manipulate the acquired ultrasound images on the computer screen. One such parameter is a bounding box or region of interest (RoI) box that, for example, may be provided in a region of a displayed ultrasound image, e.g., for Color Doppler Imaging (CDI). In such example, the RoI box facilitates visualizing blood flow in a particular portion of the ultrasound image.

Typically, when CDI is turned on, the user is presented with the RoI box on the screen, and the RoI box defines a particular region of interest. The user may adjust the position and size of the RoI box within the field of view of the ultrasound imaging by using the input devices, e.g., the track-pad or track-ball. Some imaging devices allow the user to carry out these operations directly on the display with a touch sensitive screen. However, such techniques for adjusting or otherwise controlling the RoI box are difficult to use with a handheld computing device when one hand is used to hold the ultrasound probe and the other hand is used to hold the computing device that includes the display.

### BRIEF SUMMARY

The present disclosure, in part, addresses a desire for smaller ultrasound systems, having greater portability, lower cost, and ease of use for different modes of ultrasound imaging, while at the same time providing user-friendly control and adjustment of various parameters of displayed ultrasound images. Such parameters may include, for example, the position and size of a region of interest box in Color Doppler Imaging, the range gate position in Pulse Wave Doppler imaging, the M-line position in M-Mode, the zoom of a displayed B-mode ultrasound image, or any other adjustable parameters associated with a displayed ultrasound image.

In various embodiments of systems and methods provided herein, a handheld or portable computing device is utilized as a display device for displaying ultrasound images, and includes one or more motion sensors that sense motion of the computing device. In various embodiments provided herein, the computing device utilizes such motion sensors to sense the motion and/or angular position of the computing device, and then adjusts one or more parameters of the displayed ultrasound images based on the sensed motion and/or angular position of the computing device.

In at least one embodiment, a system is provided that includes an ultrasound probe and a computing device coupled to the ultrasound probe. The computing device is operable to receive ultrasound signals from the ultrasound probe. The computing device includes a motion sensor that senses motion of the computing device, a display that displays ultrasound images associated with the ultrasound signals received from the ultrasound probe, and an image display controller coupled to the motion sensor and the display. The image display controller is operable to control at least one parameter associated with the displayed ultrasound images based on the sensed motion.

In another embodiment, the present disclosure provides a method that includes: displaying, on a display of a computing device, ultrasound images associated with ultrasound signals received from an ultrasound probe; sensing motion of the computing device by a motion sensor; and controlling at least one parameter associated with the displayed ultrasound images based on the sensed motion.

In yet another embodiment, the present disclosure provides a method that includes: displaying, on a display of a computing device, ultrasound images associated with ultrasound signals received from an ultrasound probe; receiving a first user input via the computing device; activating a motion-based control mode of the computing device in response to receiving the first user input; sensing, by a motion sensor, motion of the computing device in the motion-based control mode; and controlling at least one parameter associated with the displayed ultrasound images based on the sensed motion in the motion-based control mode.

### BRIEF DESCRIPTION OF THE SEVERAL VIEWS OF THE DRAWINGS

FIG. 1 is a schematic illustration of an ultrasound imaging device, in accordance with one or more embodiments of the present disclosure.

FIG. 2 is a block diagram illustrating components of the ultrasound imaging device, in accordance with one or more embodiments of the present disclosure.

FIG. 3 is pictorial diagram illustrating three axes of rotation of a computing device, in accordance with one or more embodiments of the present disclosure.

FIG. 4 is a pictorial diagram illustrating an example ultrasound image and region of interest (RoI) box displayed on a computing device, in accordance with one or more embodiments of the present disclosure.

FIGS. 5A to 5F are pictorial diagrams illustrating motion-based control of position and size of a RoI box, in accordance with one or more embodiments of the present disclosure.

FIG. 6 is a flow diagram illustrating a method of controlling a parameter of a displayed ultrasound image based on sensed motion, in accordance with one or more embodiments of the present disclosure.

### DETAILED DESCRIPTION

A portable ultrasound system may include a handheld computing device and an ultrasound probe that receives ultrasound imaging signals, e.g., ultrasound echo signals returning from a target structure in response to transmission of an ultrasound pulse or other ultrasound transmission signal. The computing device includes a display that displays ultrasound images associated with the received ultrasound imaging signals. The handheld computing device further includes one or more motion sensors that are capable of sensing or otherwise determining motion of the computing device with, e.g., three degrees of freedom. For example, the motion sensors can sense motion of the computing device with respect to three orthogonal axes. The sensed motion is utilized by an image display controller in the computing device to control one or more parameters associated with the displayed ultrasound images. For example, the image display controller may control a position and/or a size of a region of interest (RoI) box that is provided within a field of view of displayed color Doppler ultrasound images.

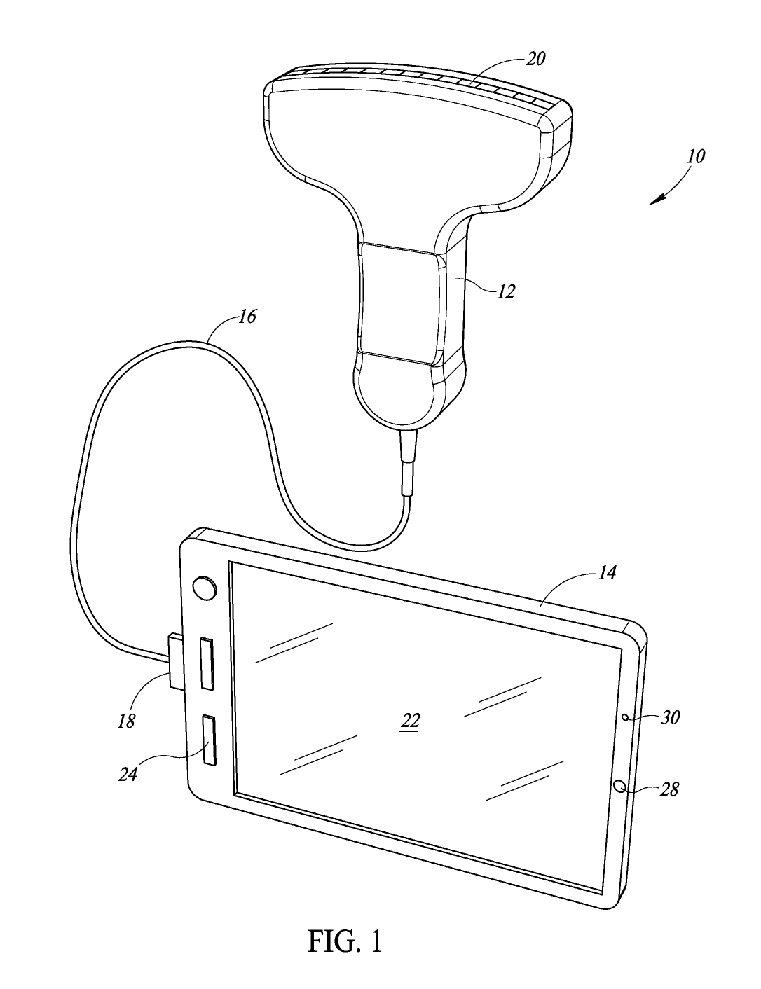

FIG. 1 is a schematic illustration of a portable ultrasound imaging device 10 (referred to herein as “ultrasound device” 10), in accordance with one or more embodiments of the present disclosure. The ultrasound device 10 includes an ultrasound probe 12 that, in the illustrated embodiment, is electrically coupled to a handheld computing device 14 by a cable 16. The cable 16 includes a connector 18 that detachably connects the probe 12 to the computing device 14. The handheld computing device 14 may be any portable computing device having a display, such as a tablet computer, a smartphone, or the like.

The probe 12 is configured to transmit an ultrasound signal toward a target structure and to receive echo signals returning from the target structure in response to transmission of the ultrasound signal. As illustrated, the probe 12 includes transducer elements 20 that are capable of transmitting an ultrasound signal and receiving subsequent echo signals.

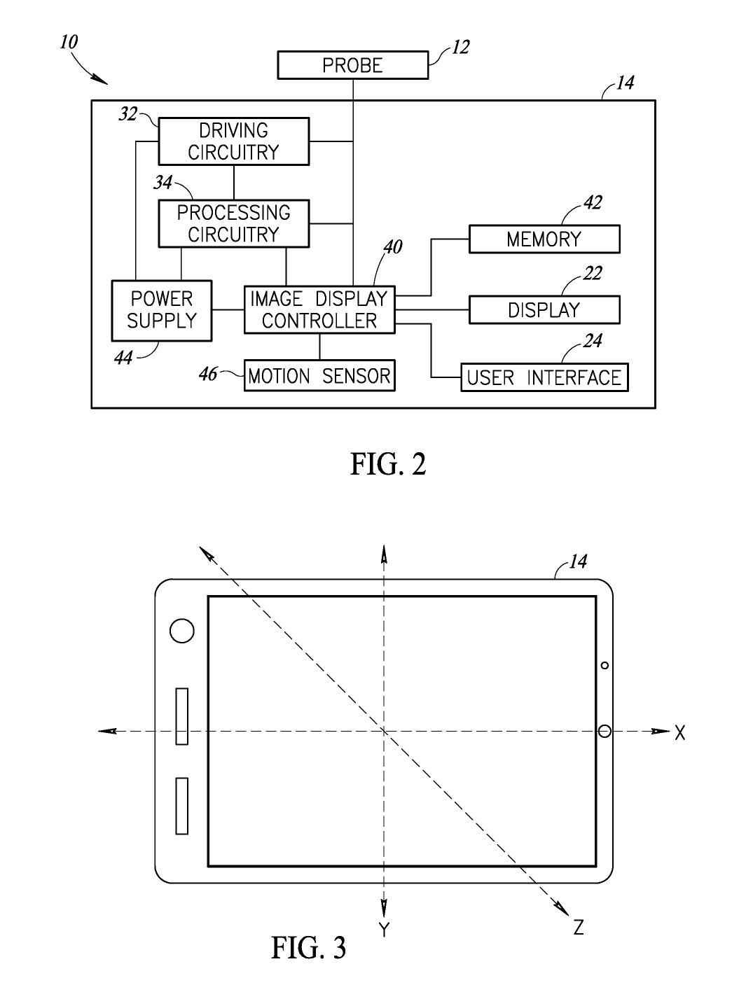

As will be described in greater detail in connection with FIG. 2, the ultrasound device 10 further includes processing circuitry and driving circuitry. In part, the processing circuitry controls the transmission of the ultrasound signal from the transducer elements 20. The driving circuitry is operatively coupled to the transducer elements 20 for driving the transmission of the ultrasound signal, .e.g., in response to a control signal received from the processing circuitry. The driving circuitry and processor circuitry may be included in one or both of the ultrasound probe 12 and the handheld computing device 14. The ultrasound device 10 also includes a power supply that provides power to the driving circuitry for transmission of the ultrasound signal, for example, in a pulsed wave or a continuous wave mode of operation.

The transducer elements 20 of the probe may include one or more transmit transducer elements that transmit the ultrasound signal and one or more receive transducer elements that receive echo signals returning from a target structure in response to transmission of the ultrasound signal. In some embodiments, some or all of the transducer elements 20 may act as transmit transducer elements during a first period of time and as receive transducer elements during a second period of time that is different than the first period of time (i.e., the same transducer elements are usable to transmit the ultrasound signal and to receive echo signals at different times).

The computing device 14 shown in FIG. 1 includes a display screen 22 and a user interface 24. The display screen 22 may be a display incorporating any type of display technology including, but not limited to, LED display technology. The display screen 22 is used to display one or more images generated from echo data obtained from the echo signals received in response to transmission of an ultrasound signal. In some embodiments, the display screen 22 may be a touch screen capable of receiving input from a user that touches the screen. In such embodiments, the user interface 24 may include a portion or the entire display screen 22, which is capable of receiving user input via touch. In some embodiments, the user interface 24 may include one or more buttons, knobs, switches, and the like, capable of receiving input from a user of the ultrasound device 10. In some embodiments, the user interface 24 may include a microphone 30 capable of receiving audible input, such as voice commands.

The computing device 14 may further include one or more audio speakers 28 that may be used to generate audible representations of echo signals or other features derived from operation of the ultrasound device 10.

FIG. 2 is a block diagram illustrating components of the ultrasound device 10, including the ultrasound probe 12 and the computing device 14. As shown in FIG. 2, the computing device 14 may include driving circuitry 32 and processing circuitry 34 for controlling and driving the transmission of an ultrasound signal from the transducer elements 20 of the ultrasound probe 12. In some embodiments, one or both of the driving circuitry 32 and processing circuitry 34 are included in the ultrasound probe 12. That is, the ultrasound probe 12 may contain the circuitry that controls the driving the transducer elements 20 to transmit an ultrasound signal, and may further include circuitry for processing received echo signals.

In various embodiments, the processing circuitry 34 includes one or more programmed processors that operate in accordance with computer-executable instructions that, in response to execution, cause the programmed processor(s) to perform various actions. For example, the processing circuitry 34 may be configured to send one or more control signals to the driving circuitry 32 to control the transmission of an ultrasound signal by the transducer elements 20 of the ultrasound probe 12.

The driving circuitry 32 may include an oscillator or other circuitry that is used when generating an ultrasound signal to be transmitted by the transducer elements 20. Such an oscillator or other circuitry may be used by the driving circuitry 32 to generate and shape the ultrasound pulses that form the ultrasound signal.

The computing device 14 further includes an image display controller 40 that provides ultrasound image information for display on the display 22. The image display controller 40 may include one or more programmed processors that operate in accordance with computer-executable instructions that, in response to execution, cause the programmed processor(s) to perform various actions. In some embodiments, the image display controller 40 may be a programmed processor and/or an application specific integrated circuit configured to provide the image display control functions described herein. The image display controller 40 may be configured to receive ultrasound signals from the processing circuitry 34 or from the ultrasound probe 12, and to generate associated ultrasound image information based on the received ultrasound signals. The ultrasound image information may be provided from the image display controller 40 to the display 22 for displaying an ultrasound image. The image display controller 40 is further configured to control one or more parameters of the displayed ultrasound image, as will be discussed in further detail herein. The image display controller 40 may be coupled to computer-readable memory 42, which may store computer-executable instructions that, in part, are executable by the image display controller 40 and cause the image display controller 40 to perform the various actions described herein.

In one or more embodiments, the processing circuitry 34 and the image display controller 40 may be fully or partially combined, such that the features and functionality of the processing circuitry 34 and the image display controller 40 are provided by one or more shared processors.

For example, in one or more embodiments, the image display controller 40 may be included in, or executed by, the processing circuitry 34. The image display controller 40 may be a module executed by one or more processors included in the processing circuitry 34. In other embodiments, the image display controller 40 may be configured with processing circuitry separate from the processing circuitry 34 and may operate in cooperation with the processing circuitry 34.

The image display controller 40 is coupled to the user interface 24. The user interface 24 may receive user input, for example, as touch inputs on the display 22, or as user input via one or more buttons, knobs, switches, and the like. In some embodiments, the user interface 24 may receive audible user input, such as voice commands received by a microphone 30 of the computing device 14. The image display controller 40 is configured to provide the ultrasound image information, and to control the parameters of the ultrasound images displayed on the display 22, based on user input received by the user interface 24.

The processing circuitry 34 and/or the image display controller 40 may control a variety of operational parameters associated with the driving circuitry 32, the display 22 and the user interface 24.

The computing device 14 includes a power supply 44 that is electrically coupled to various components the computing device 14. Such components may include, but are not limited to, the processing circuitry 34, the driving circuitry 32, the image display controller 40, the display 22, the interface 24, and any other components of the computing device 14 illustrated in FIG. 2. The power supply 44 may provide power for operating the processing circuitry 34 and the driving circuitry 32. In particular, the power supply 44 provides power for generating the ultrasound signal by the driving circuitry 32 and transmitting the ultrasound signal, with stepped-up voltage as needed, by the transducer elements 20. The power supply 44 may also provide power for the driving circuitry 32 and the processing circuitry 34 when receiving echo signals, e.g., via the transducer elements 20. The power supply 44 may further provide power for the display 22 and the user interface 24. The power supply 44 may be or include, for example, one or more batteries in which electrical energy is stored and which may be rechargeable.

The computing device 14 further includes one or more motion sensors 46 coupled to the image display controller 40. The image display controller 40 is operable to control one or more parameters associated with the displayed ultrasound image based on motion of the computing device 14 sensed by the motion sensors 46, as will be described in further detail below.

The motion sensor 46 may include, for example, one or more accelerometers, gyroscopes, or combinations thereof for sensing motion of the computing device 14. For example, the motion sensor 46 may be or include any of a piezoelectric, piezoresistive or capacitive accelerometer capable of sensing motion of the computing device 14, preferably in three dimensions. In one or more embodiments, the motion sensor 46 is a three-axis accelerometer or other suitable motion sensor that is capable of sensing translational or rotational motion of the computing device 14 along or about any of three orthogonal axes (e.g., x-axis, y-axis, and z-axis).

The motion sensor 46 may be any sensor that can be used to sense, detect, derive or determine motion of the computing device 14. In some embodiments, the motion sensor 46 does not itself sense motion, but instead may be a sensor that outputs signals from which motion of the computing device 14 can be derived. For example, in one or more embodiments, the motion sensor 46 may be one or more cameras, including 2D and/or 3D cameras, and in other embodiments, the motion sensor 46 may be one or more optical sensors. The signals output by such cameras and/or optical sensors can be processed using any signal processing techniques suitable to determine relative motion of the computing device 14 based on the output signals. For example, optical flow methods may be implemented to determine relative motion of the computing device 14 based on an apparent motion or displacement of image objects between consecutive frames acquired by a camera. In some embodiments, the motion sensor 46 may include one or more cameras or optical sensors which, in combination with one or more spatial models (e.g., as may be employed in Augmented Reality techniques), can be used to derive relative motion of the computing device 14 through 2D or stereo images of the surroundings. Accordingly, as used herein, the term “sensed motion” includes sensing signals from which motion may be determined and/or derived, and includes, for example, output signals from a 2D or 3D camera or an optical sensor, which may be utilized to determine a motion of the computing device 14.

During operation of the ultrasound device 10, the ultrasound probe 12 acquires ultrasound signals, e.g., echo signals returning from the target structure in response to a transmitted ultrasound signal. The echo signals may be provided to the processing circuitry 34 and/or the image display controller 40, either or both of which may include ultrasound image processing circuitry for generating ultrasound image information based on the received echo signals. Such ultrasound image processing circuitry may include, for example, amplifiers, analog-to-digital converters, delay circuitry, logic circuitry, and the like, which is configured to generate ultrasound image information based on the received echo signals.

The ultrasound image information is provided to the image display controller 40, which generates or otherwise outputs ultrasound images associated with the received ultrasound signals to the display 22 for displaying the ultrasound images. Such ultrasound images may be ultrasound images associated with any of a variety of ultrasound imaging modes, such as A-mode (amplitude mode), B-mode (brightness mode), M-mode (motion mode), Doppler mode (including Color Doppler, Continuous Wave (CW) Doppler, and Pulsed Wave (PW) Doppler), and so on. Moreover, the ultrasound images may be 2D, 3D, or 4D ultrasound images.

The image display controller 40 may include various modules and/or circuitry configured to extract relevant components from the received ultrasound image information for any of the ultrasound imaging modes. The ultrasound imaging mode may be a selectable feature, such that a user may select a particular imaging mode, and the image display controller 40 will output ultrasound images to the display 22 that are associated with the selected mode. Depending on the selected ultrasound imaging mode, the sensed motion of the computing device 14 may control different parameters associated with the displayed ultrasound images.

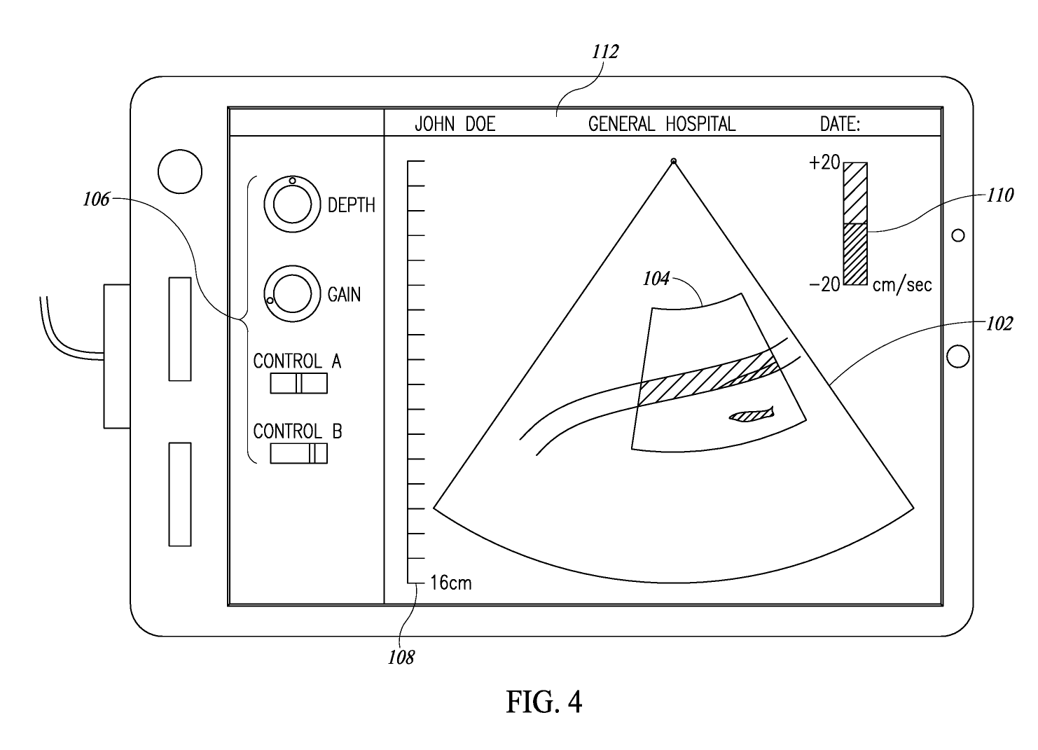

FIG. 3 is pictorial diagram illustrating three orthogonal axes of rotation of a computing device, in accordance with one or more embodiments of the present disclosure. The motion sensor 46 is operable to sense motion of the computing device 14 relative to each of the axes illustrated in FIG. 3, namely, each of the x-axis, y-axis, and z-axis. In operation, the image display controller 40 receives signals indicative of the motion of the computing device 14 from the motion sensor 46. Such signals indicative of the motion of the computing device 14 may be signals received from a motion sensor such as an accelerometer or gyroscope, and/or may be signals from which motion of the computing device 14 may be derived or otherwise determined, such as signals received from one or more cameras or optical sensors.

The image display controller 40 may include or be communicatively coupled to signal processing circuitry or modules that performs processing, filtering, tuning, or the like on the signals indicative of the motion of the computing device 14 to transform the signals into a control input for controlling one or more parameters of an ultrasound image. The image display controller 40 may thus dynamically control a parameter of an ultrasound image that is prepared by the computing device 14 for displaying on the display 22 based on the sensed motion. The sensed motion of the computing device 14 relative to each of the x-axis, y-axis, and z-axis may be, for example, translational motion having vector components along one or more of the axes or rotational motion about one or more of the axes.

In one or more embodiments, the image display controller 40 controls parameters related to a region of interest (RoI) box in a Color Doppler imaging (CDI) mode based on the sensed motion of the computing device 14.

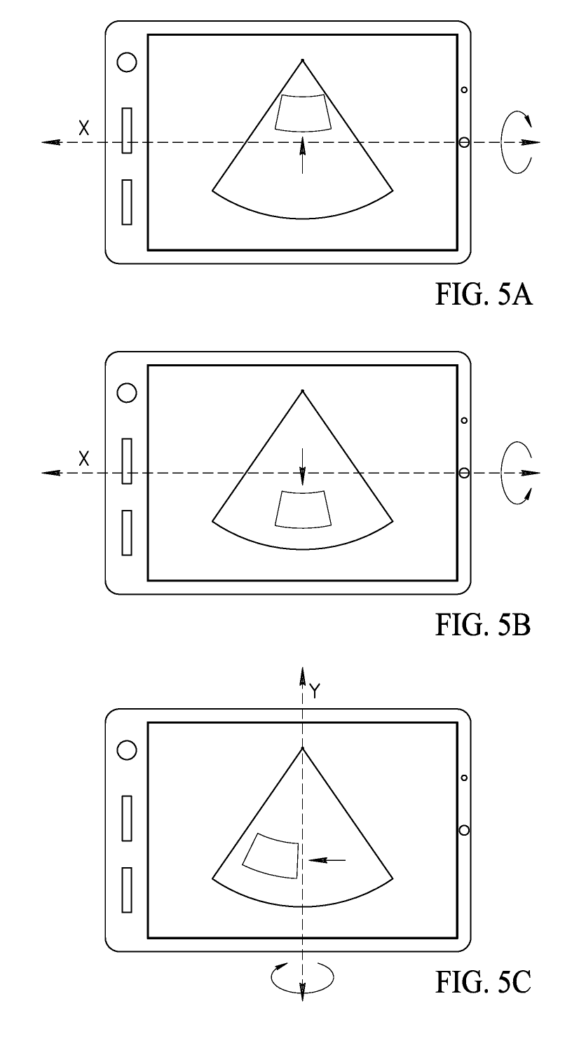

FIG. 4 is a pictorial diagram showing an example ultrasound image 102 and RoI box 104 displayed on the computing device 14, in accordance with one or more embodiments. Various other features may be displayed concurrently with the ultrasound image 102, including, for example, controls 106, imaging scale 108, color flow scale 110, and clinical information 112. The controls 106 may be user-controllable features that are displayed on the display 22, and the provided controls 106 may depend on the selected imaging mode. For example, as shown in FIG. 4, the controls 106 for CDI mode imaging may include depth control, gain control, and various other controls such as Control A and Control B. The imaging scale 108 may be a 2D B-mode depth scale in B-mode and in CDI mode imaging. The color flow scale 110 may be displayed in the CDI mode, and provides a color-coded scale that indicates flow velocities based on the colors displayed in the RoI box 102. The clinical information 112 may include, for example, a patient name, a clinic or hospital name, and an imaging date.

In the following description, the control of a size and/or position of the RoI box 104 in CDI mode is described as an example of motion-based control of a parameter associated with a displayed ultrasound image, in accordance with one or more embodiments of the present disclosure. However, embodiments of the present disclosure are not limited to controlling size and/or position of a RoI box 104 in CDI mode. Any parameter of a displayed ultrasound image may be controlled based on sensed motion in accordance with embodiments of the present disclosure, including, for example, a range gate position in Pulse Wave Doppler imaging, an M-line position in M-Mode, a zoom of a displayed B-mode ultrasound image, or any other adjustable parameters associated with a displayed ultrasound image.

As shown in FIG. 4, the displayed ultrasound image 102 represents a field of view acquired by the ultrasound probe 12 in the CDI mode. More particularly, the ultrasound image 102 corresponds with a 2-dimensional B-mode ultrasound image, and a Color Doppler RoI box 104 is overlaid on a portion of the B-mode image within the field of view. Within the RoI box 104, velocity information, such as velocity information related to blood flow, is presented in a color-coded scheme. The RoI box 104 may be provided in the ultrasound image 102 field of view upon entry of the ultrasound device 14 into the CDI mode. For example, the ultrasound device 10 may initially be imaging in the B-mode, and then the user may turn on the CDI mode, which causes the RoI box 104 to appear within the field of view of the displayed ultrasound image 102.

In some embodiments, when the CDI mode is entered, the RoI box 104 is presented at a default position within the field of view of the ultrasound image 102. For example, the default position may be located in a center region of the field of view of the ultrasound image 102. In other embodiments, the RoI box 104 may initially be presented at a position within the field of view of an ultrasound image 102 that corresponds with a previous position of the RoI box 104, e.g., as last set by the user.

In one or more embodiments, the user may selectively enter the CDI mode, e.g., from the B-mode, by user input via the user interface 24. For example, CDI mode may be entered by pressing a physical button on the computing device 14 or by pressing a virtual button, e.g., as may be presented on the touchscreen of the display 22. In some embodiments, the CDI mode may be entered by pressing and holding such buttons for a threshold period of time, and in other embodiments, the CDI mode may be entered by simply tapping a button or by tapping the display 22. In some embodiments, the CDI mode may be entered by providing a suitable voice command.

Once the CDI mode is entered, the RoI box 104 is presented within the field of view of the ultrasound image 102, for example, at the default or last-used position. Motion-based control of the RoI box 104 may be automatically activated upon entering the CDI mode in some embodiments, and in other embodiments, additional user input may be needed in order to activate the motion-based control. Such additional user input may include user input provided via the user interface 24, including user input provided by pressing or pressing and holding one or more physical or virtual buttons, a touch on the touchscreen display 22, a voice command, or the like.

Once motion-based control of the RoI box 104 is activated, one or more parameters of the RoI box 104 is controlled based on motion of the computing device 14 sensed by the motion sensor 46. In particular, the image display controller 40 receives signals from the motion sensor 46 indicative of the sensed motion of the computing device 14 and may control a position and/or a size of the RoI box 104 based on the sensed motion.

As soon as motion-based control is activated, whether automatically upon entry of CDI mode or by additional user input, the position and/or orientation of the computing device 14 at the time of activation of motion-based control may be used as an initial position and/or orientation for motion sensing purposes. Accordingly, any motion of the computing device 14 along or about any of the orthogonal x-axis, y-axis, and z-axis may be determined with respect to the initial position and/or orientation of the computing device 14. Alternatively, or additionally, motion of the computing device 14 along or about any of the orthogonal x-axis, y-axis, and z-axis may be determined relative to a previously determined position and/or orientation of the computing device 14.

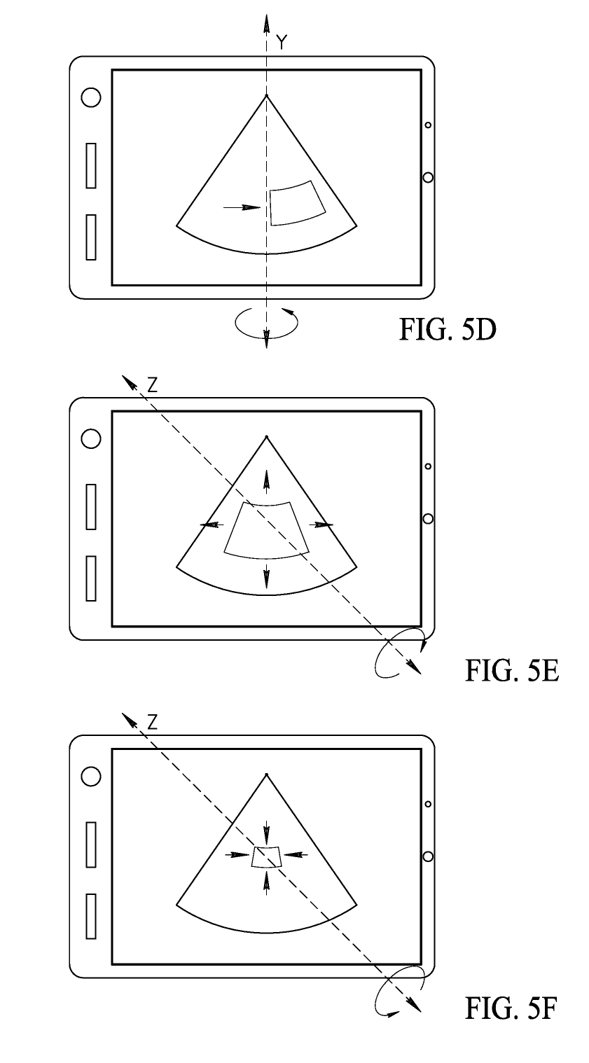

As noted earlier herein, the sensed motion of the computing device 14 may be translational motion along, or rotational motion about, any of the x-axis, y-axis, and z-axis. The sensed motion relative to each respective axis may be used by the image display controller 40 to adjust a particular parameter of the RoI box 104. For example, in some embodiments, the sensed motion is used by the image display controller 40 to adjust a position of the RoI box 104 within the field of view of the ultrasound image 102, as shown in FIGS. 5A to 5D.

As shown in FIG. 5A, the image display controller 40 moves the position of the RoI box 104 up with respect to the field of view of the ultrasound image 102 in response to rotation of the computing device 14 about the x-axis in a first direction (e.g., tilting the computing device 14 back). As shown in FIG. 5B, the image display controller 40 moves the RoI box 104 down in response to rotation of the computing device 14 about the x-axis in a second direction that is opposite to the first direction (e.g., tilting the computing device 14 forward).

As shown in FIG. 5C, the image display controller 40 moves the position of the RoI box 104 to the left in response to rotation of the computing device 14 about the y-axis in a first direction (e.g., tilting the computing device 14 to the left). As shown in FIG. 5D, the image display controller 40 moves the position of the RoI box 104 to the right in response to rotation of the computing device 14 about the y-axis in a second direction that is opposite to the first direction (e.g., tilting the computing device 14 to the right).

The motion sensor 46 can sense motion along or about multiple axes concurrently. Accordingly, the RoI box 104 can be repositioned by moving the RoI box 104 within the field of view of the ultrasound image 102 in directions that are between two or more of the axial directions. For example, tilting the computing device 14 back (i.e., rotating the computing device 14 in a first direction about the x-axis) and to the right (i.e., rotating the computing device 14 in a second direction about the y-axis) at the same time will cause the image display controller 40 to move the RoI box 104 in a direction that is both up and to the right at the same time.

In some embodiments, the size of the RoI box 104 relative to the size of the displayed ultrasound image 102 is adjustable based on the sensed motion of the computing device 14, as shown in FIGS. 5E and 5F.

As shown in FIG. 5E, the image display controller 40 may increase the size of the RoI box 104 in response to rotation of the computing device 14 about the z-axis in a first direction. The size of the RoI box 104 may be increased by extending the boundaries of the RoI box 104 proportionally outwardly about a center point of the RoI box 104.

As shown in FIG. 5F, the image display controller 40 may decrease the size of the RoI box 104 in response to rotation of the computing device 14 about the z-axis in a second direction that is opposite to the first direction. The image display controller 40 may decrease the size of the RoI box 104 by proportionally contracting the boundaries of the RoI box 104 inwardly toward the center point of the RoI box 102.

The control of the position and the size of the RoI box 104 are shown in FIGS. 5A to 5F as being based on rotations about the x-axis, y-axis, and z-axis; however, it should be readily appreciated that in various embodiments, the position and/or size of the RoI box 104 may be similarly controlled based on translational motion along any of the x-axis, y-axis, and z-axis.

In some embodiments, the adjustable parameters of the RoI box 104 may be selectively turned on and off, such that a particular parameter of the RoI box 104 will not be changed when that parameter is not turned on or otherwise active, even though the computing device 14 may be moved along or about the axis that normally causes the particular parameter of the RoI to be adjusted. For example, in one or more embodiments, a user may activate motion-based control of the position of the RoI box 104, while the size of the RoI box 104 remains fixed. In such embodiments, the user may enter the Color Doppler Imaging mode, e.g., by pressing or pressing and holding a button of the user interface 24, by tapping the touchscreen display 22, by a voice command, or the like, as previously discussed herein. Motion-based control of the position of the RoI box 104 may automatically commence upon entry of the CDI mode, or in various embodiments, motion-based control of the position of the RoI box 104 may be commenced upon another user input, such as pushing a button, tapping the touchscreen, a voice command or the like.

The user may thus control the position of the RoI box 104, for example, by translational or rotational movement along or about the x-axis and the y-axis. Motion of the computing device 14 along or about the z-axis will not change the size of the RoI box 104, since motion-based control based on the z-axis has not been activated or otherwise turned on. The user may selectively activate control of the size of the RoI box 104, based on motion of the computing device 14 along or about the z-axis, by providing additional user input. For example, the user may activate motion-based control of the size of the RoI box 104 by pushing a button, releasing a previously held button, tapping the touchscreen display 22, providing a suitable voice command, or the like.

Accordingly, in some embodiments, the position and the size of the RoI box 104 may be concurrently adjustable based on motions about any of the x-axis, y-axis, and z-axis. And, in some embodiments, adjustment of the position and the size of the RoI box 104 may be provided by independent motion-based control modes that are selectively entered by the user.

In some embodiments, the user may enter the motion-based control mode, in which the RoI box 104 or other parameter is controlled based on the sensed motion of the computing device 14, by pressing and holding a physical or virtual button of the user interface 24. The motion-based control mode may be activated for only a time that the user continues to hold the button. When the user releases the button, the motion-based control mode may be deactivated, and the RoI box 104 may be displayed with a position and/or size as produced at the time of deactivation of the motion-based control mode. This allows the RoI box 104 to be “locked” at a desired position when the user releases the button, and the user may then set the computing device 14 down, e.g., on a table or in a tablet holder on an ultrasound cart, while the user continues to hold the probe 12 for ultrasound imaging. Similarly, in some embodiments, the RoI box 104 may be “locked” in place by an additional user input, such as pressing a physical or virtual button, or by a touch input on the touchscreen display 22.

In some embodiments, the position and/or size of the RoI box 104 may be “locked” in response to the computing device 14 being relatively motionless for some threshold period of time, e.g., for 1 or 2 seconds. For example, if the motion sensor 46 detects no motion or only insignificant motion (as may be determined based on some threshold value of motion) for some period of time, then the computing device 14 may fix the RoI box 104 at its current size and position.

When the RoI box 104 is “locked” in a particular size and/or position, the ultrasound device 10 may continue to image a target and the field of view of the displayed ultrasound images may change, for example, by the moving the probe 12. However, in the locked state, the RoI box 104 will remain in a fixed position with respect to the displayed field of view, regardless of changes in the field of view.

Motion-based control of a parameter of a displayed ultrasound image, as provided in various embodiments herein, allows for convenient control of parameters, such as position and/or size of the RoI box of a displayed ultrasound image. Such motion-based control may be particularly convenient and advantageously utilized by users of ultrasound systems that include a handheld computing device. In particular, the user of such a handheld computing device can manipulate the RoI box (or other parameter, depending on application) using just one hand. For example, the user may hold the probe 12 in one hand, and may hold the computing device 14 in the other hand. The hand that is holding the computing device 14 may also be used to provide user input (e.g., by a thumb or a finger) while holding the computing device 14, and the user input can initiate the motion-controlled features of the present disclosure. Once activated, the user can move and/or resize the RoI box as desired, all while holding the probe 12 in one hand and the computing device 14 in the other hand.

In some embodiments, one or more operational parameters of the driving circuitry 32 and/or the processing circuitry 34 may be controlled or adjusted based on the sensed motion of the computing device 14. For example, in some embodiments, the sensed motion of the computing device 14 is used to control a displayed parameter such as a range gate in Pulse Wave Doppler imaging. In such a case, a change in the motion of the computing device 14 changes the range within which echo signals are measured, which may be changed by the processing circuitry 32 that acquires or measures the echo signals. More particularly, changing the range gate may change a listening region within a sampled volume from which the returning echo signals are accepted. The width and height of the range gate is determined by the width and height of the transmitted ultrasound beam, and the length of the range gate is determined by the pulse length of the transmitted beam. Accordingly, motion-based control or adjustments of the range gate of the displayed ultrasound image may involve concurrent control of the driving circuitry 32 and/or the processing circuitry 34 in order to transmit and receive a suitable ultrasound signal for the adjusted range gate.

In one or more embodiments, motion-based control of a parameter of a displayed ultrasound image, such as motion-based control of a RoI box, as described herein may be provided as an additional or alternative mode for controlling the parameter. For example, in some embodiments, the size and position of the RoI box may be adjustable based on user inputs provided, e.g., from a peripheral input device such as a mouse, a touchpad of a laptop computer, a keyboard, or the like. The computing device 14 may additionally be configured to adjust the RoI box in a motion-based control mode, in which the RoI box is controlled based on sensed motion of the computing device 14, as described herein. In such embodiments, a user may selectively activate the motion-based control or the user input-based control of the RoI box. The user may, for example, activate use input-based control of the RoI box, in which the size and/or position of the RoI box is adjustable based on user inputs, when the computing device 14 is stationary, such as when mounted on an ultrasound cart or docked in a docking station. However, when the user wishes to hold the computing device 14 while imaging with the ultrasound probe 12, the user may activate the motion-based control mode so that the RoI box may be manipulated based on the motion of the computing device 14.

Embodiments provided herein are not limited to direct proportional control between the signals indicative of the motion of the computing device 14 and the controlled parameter. Instead, as discussed previously herein, the image display controller 40 may include or be communicatively coupled to signal processing circuitry or modules that processes the signals indicative of the motion of the computing device 14 to generate a control input for controlling one or more parameters of an ultrasound image. The image display controller 40 may thus blend, filter, tune, or further process multiple signals from one or more sensors in order to control the one or more parameters. For example, an accelerometer output signal indicating motion of the computing device 14 (e.g., a signal associated with the user quickly wiggling or moving the computing device 14 to the left) may be processed and utilized to move the ROI box 104 one unit (e.g., one grid step) to the left. In such a case, the accelerometer output signal may be processed using signal processing techniques such as a comparison with one or more thresholds, filtering out spurious signals or signals indicative of unintended motion, or the like, to transform a continuous accelerometer reading into a binary movement (e.g., one unit left), which is then used to control the RoI box 104.

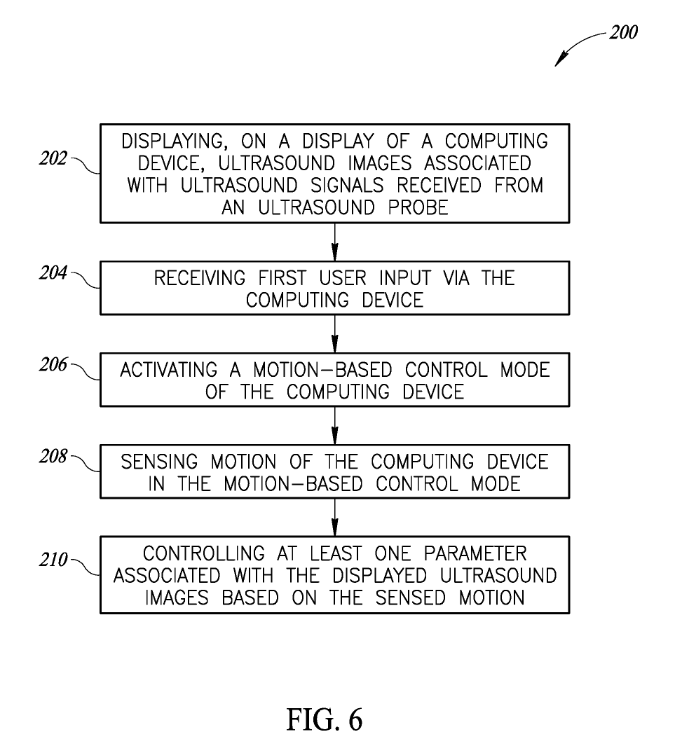

FIG. 6 is a flow diagram illustrating a method 200, in accordance with one or more embodiments of the present disclosure. In at least one embodiment, the method 200 includes, at block 202, displaying, on a display 22 of a computing device 14, ultrasound images associated with ultrasound signals received from an ultrasound probe 12. The received ultrasound images may be ultrasound images associated with any ultrasound imaging mode, e.g., B-mode, M-mode, Color Doppler mode, Pulsed Wave Doppler mode, and the like.

At block 204, the method 200 includes receiving a first user input via the computing device 14. The first user input may be provided, for example, through the user interface 24 of the computing device 14, which may include user input provided via pressing or pressing and holding a physical or virtual button, one or more touches on a touch screen of the display 22, voice commands provided via the microphone 30, or the like.

At block 206, the method 200 includes activating a motion-based control mode of the computing device 14. The motion-based control mode may be activated in response to receiving the first user input.

At block 208, the method 200 includes sensing motion of the computing device 14 in the motion-based control mode. The motion of the computing device 14 is sensed, for example, by the motion sensor 46.

At block 210, the method 200 includes controlling at least one parameter associated with the displayed ultrasound images based on the sensed motion in the motion-based control mode. The at least one parameter may include, for example, a position and/or a size of a region of interest box 104 within a field of view of a displayed ultrasound image 102 in a color Doppler imaging mode.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A system, comprising:
an ultrasound probe;
a computing device coupled to the ultrasound probe and operable to receive ultrasound signals from the ultrasound probe, the computing device including:
a motion sensor that senses motion of the computing device;
a display that displays ultrasound images associated with the ultrasound signals received from the ultrasound probe; and
an image display controller coupled to the motion sensor and the display, the image display controller being operable to control at least one parameter associated with the displayed ultrasound images based on the sensed motion.

2. The system of claim 1 wherein the ultrasound images are color Doppler ultrasound images, and the at least one parameter includes a region of interest box associated with the color Doppler ultrasound images.

3. The system of claim 2 wherein the image display controller is operable to control a position of the region of interest box within a field of view of the color Doppler ultrasound images based on the sensed motion.

4. The system of claim 3 wherein the image display controller is operable to control a size of the region of interest box within the field of view of the color Doppler ultrasound images based on the sensed motion.

5. The system of claim 1 wherein the motion sensor includes at least one of an accelerometer, a gyroscope, a 2D camera, a 3D camera, and an optical sensor.

6. The system of claim 1 wherein the motion sensor includes an accelerometer operable to sense translational or rotational motion of the computing device along or about at least one sensing axis, and the image display controller is operable to control at least one parameter associated with the displayed ultrasound images based on the sensed translational or rotational motion.

7. The system of claim 1 wherein the motion sensor includes a three-axis accelerometer operable to sense translational or rotational motion of the computing device along or about each of three orthogonal axes, and the image display controller is operable to control three separate parameters associated with the displayed ultrasound images, each of the three separate parameters being controllable by the image display controller based on sensed motion of the computing device along or about a respective one of the three orthogonal axes.

8. The system of claim 6 wherein the ultrasound images are color Doppler ultrasound images, and the three separate parameters include a position of a region of interest box associated with the color Doppler ultrasound image along a first direction, a position of the region of interest box along a second direction that is orthogonal to the first direction, and a size of the region of interest box.

9. The system of claim 1 wherein the computing device further includes a user interface operable to receive user input, and the image display controller is operable to selectively enter a motion-based control mode based on user input received via the user interface, wherein the image display controller is operable to control the at least one parameter associated with the displayed ultrasound images only when in the motion-based control mode.

10. The system of claim 9 wherein the computing device is operable to determine an initial position of the computing device upon entry of the motion-based control mode, and the image display controller is operable to control the at least one parameter based on sensed motion of the computing device with respect to the initial position.

11. The system of claim 9 wherein the image display controller is operable to selectively enter the motion-based control mode based on a first user input received via the user interface and to control a first parameter associated with the displayed ultrasound images based on the sensed motion in the motion-based control mode, and the image display controller is operable to control a second parameter associated with the displayed ultrasound images based on a second user input received via the user interface.

12. The system of claim 11 wherein the ultrasound images are color Doppler ultrasound images, the first parameter includes a position of a region of interest box associated with the color Doppler ultrasound images, and the second parameter includes a size of the region of interest box.

13. A method, comprising:
displaying, on a display of a computing device, ultrasound images associated with ultrasound signals received from an ultrasound probe;
sensing motion of the computing device by a motion sensor; and
controlling at least one parameter associated with the displayed ultrasound images based on the sensed motion.

14. The method of claim 13 wherein the ultrasound images include color Doppler ultrasound images, and controlling the at least one parameter includes controlling at least one of: a position of a region of interest box within a field of view of the color Doppler ultrasound images, and a size of the region of interest box within the field of view of the color Doppler ultrasound images.

15. The method of claim 13 wherein sensing motion of the computing device by the motion sensor includes sensing translational or rotational motion of the computing device along or about each of three orthogonal axes, and controlling the at least one parameter includes controlling three parameters associated with the displayed ultrasound images, each of the three parameters being controlled based on sensed motion of the computing device along or about a respective one of the three orthogonal axes.

16. A method, comprising:
displaying, on a display of a computing device, ultrasound images associated with ultrasound signals received from an ultrasound probe;
receiving a first user input via the computing device;
activating a motion-based control mode of the computing device in response to receiving the first user input;
sensing, by a motion sensor, motion of the computing device in the motion-based control mode; and
controlling at least one parameter associated with the displayed ultrasound images based on the sensed motion in the motion-based control mode.

17. The method of claim 16 wherein receiving the first user input includes receiving user input information associated with pressing and holding a button, the method further comprising:
receiving a second user input, the second user input information associated with releasing the button; and
deactivating the motion-based control mode based on the received second user input.

18. The method of claim 16, further comprising:
receiving a second user input via the computing device in the motion-based control mode; and
controlling at least one other parameter associated with the displayed ultrasound images based on the sensed motion of the computing device.

19. The method of claim 18 wherein the ultrasound images include color Doppler ultrasound images, controlling the at least one parameter includes controlling a position of a region of interest box within a field of view of the color Doppler ultrasound images, and controlling the at least one other parameter includes controlling a size of the region of interest box within the field of view of the color Doppler ultrasound images.

20. The method of claim 16, wherein the ultrasound images include color Doppler ultrasound images, and controlling the at least one parameter includes controlling a position of a region of interest box within a field of view of the color Doppler ultrasound images, the method further comprising:
receiving a second user input via the computing device in the motion-based control mode; and
fixing the position of the region of interest box within the field of view of the color Doppler ultrasound images in response to receiving the second user input.
